(19) United States
(12) Patent Application Publication
(10) Pub. No.: US 2010/0173290 A1
(43) Pub. Date: Jul. 8, 2010

(54) MONITORING OF ENZYMATIC PROCESSES BY USING MAGNETIZABLE OR MAGNETIC OBJECTS AS LABELS

(75) Inventor: Menno Willem Jose Prins, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/377,167

(22) PCT Filed: Aug. 10, 2007
(86) PCT No.: PCT/IB07/53184

§ 371 Date: Feb. 11, 2009

(30) Foreign Application Priority Data
Aug. 14, 2006 (EP) 06118873.6

(51) Int. Cl. C12Q 1/68; C12Q 1/00; C12M 1/00

(52) U.S. Cl. 435/6; 435/4; 435/287.1

(57) ABSTRACT

The present invention provides a method and device for monitoring an enzymatic process of a biological molecule by, at least once, during the enzymatic process determining an amount of magnetic particles attached to a sensor surface. The method and device according to embodiments of the present invention may be used for monitoring an enzymatic process as a function of time.

### FIELD OF THE INVENTION

The present invention relates to enzymatic processes and more particularly to a method and system for monitoring an enzymatic process on a biological molecule using magnetizable or magnetic objects as labels. The method and device according to the present invention may be used for monitoring an enzymatic process as a function of time.

### BACKGROUND OF THE INVENTION

Nucleic acids (RNA, DNA) can be very sensitively and specifically measured by using a biochemical amplification process. Nucleic-acid amplification and subsequent detection are complicated processes that generally require several process steps. For the detection of biological material (e.g. micro-organisms) these steps include typically (selective) enrichment, isolation/purification and identification. There are large efforts ongoing to simplify the processes and improve the analytical performance (e.g. sensitivity, specificity, speed). For example, it has been shown that it is feasible to sensitively and specifically detect the products of RNA/DNA amplification by means of ligand proteins through a combination of molecular biological techniques and immuno-detection. For signal development other assays use fluorescence, chemiluminescence or a wide variety of (immuno)sensor detectors instead of colorimetry. However, most of the assays developed are relatively complex, expensive and/or require (sophisticated) instrumentation.

A simplified extraction and amplification method combined with a lateral flow immunoassay (LFIA) detection method has been developed, which is several orders of magnitude more sensitive than gel electrophoresis and results are revealed within 5 to 15 minutes. In principle, LFIA is suited for multi-analyte detection, i.e., the screening for up to 6 specific RNA/DNA sequences in one assay device. A method based on black colloidal particles and specific ligands immobilised on nitrocellulose membranes enables the detection of 5 to 30 different parameters in a mini-array set up. The results can be digitised by flatbed scanning and image analysis.

The quantitative aspect and the dynamic range of the measurement is an important problem in biochemical amplification. This is particularly the case for exponential amplification methods such as PCR, which have a steep transition between sub-detection-level amplicon concentrations on the one hand and saturation of the biochemical amplification process on the other hand. Often the result is a yes-or-no answer rather than a precise value for the target concentration.

An improved method is Real-time PCR, wherein the concentration of amplicons is dynamically measured (e.g. with molecular beacons) during the exponential biochemical amplification process. In quantitative real-time PCR, quantitative data are derived using dedicated probe design, process control and process monitoring. The original target concentration is deduced from the time required to develop a certain signal. Disadvantages of quantitative real-time PCR are (i) the complicated assay procedure, (ii) the high cost-level per test, and (iii) the difficulty to perform assay-multiplexing.

In their most commonly used forms, the above methods (ELISA, LFIA, real-time PCR) all involve optical detection. Optical detection can have several disadvantages, such as

High background signals (e.g. autofluorescence from the substrate or the device material, from the sample material or from the biological materials in the test device) particularly when complex biological samples are used.

Label properties can depend on the biochemical environment (e.g. fluorescence efficiency), which complicates quantification of the measurement

Error-prone interconnections between cartridge and reader.

Light scattering from the device and from the fluid sample.

Absorption of incident and emitted/reflected light depends on the optical properties of the fluid.

Sometimes expensive readout equipment is needed.

Alternative detection methods to light-based detection exist. For example magnetic sensors which detect magnetic nanoparticles are being investigated for bio-diagnostic purposes due to the following expected advantages: high analytical performance (sensitivity (biological materials have a very low magnetic background and very sensitive sensors are available)), speed (due to magnetic actuation), specificity (due to force discrimination), combined assay steps (e.g. target extraction and detection, both with magnetic particles), and easy to use (simple and reliable electrical interconnect, the sensor and reader are compact and of low cost).

Preliminary attempts have been made to combine nucleic-acid amplification with magnetic detection. WO00/61803 discloses a combination of nucleic-acid amplification followed by detection on a magnetic sensor chip. The process includes the application of stringency by magnetic forces. The solution is particularly focused on the method of Strand Displacement Amplification (SDA). EP0781346 discloses a method wherein PCR amplification is alternated with a magnetic detection method. The magnetic detection is based on the difference in migration between magnetically labelled primers and amplified DNA.

In view of the drawbacks of the previous systems, there is a need for rapid, sensitive, quantitative, high dynamic range, and real-time nucleic-acid detection processes based on alternative detection methods and/or alternative labels and/or alternative sensors. Such methods should contain as few steps as possible, i.e. have maximum integration of biochemical processes and detection.

### SUMMARY OF THE INVENTION

The present invention provides a method and system for monitoring an enzymatic process. The method comprises:

allowing an enzymatic process to take place in a reaction chamber, the reaction chamber being provided with magnetizable or magnetic objects such as particles,

providing a sensor device having a sensor surface,

during the enzymatic process, subsequently attracting and withdrawing the magnetizable or magnetic objects to and from the sensor surface, and

at least once during the enzymatic process, after attracting the magnetizable or magnetic objects to the sensor surface, measuring the amount of magnetizable or magnetic objects attached to the sensor surface.

According to embodiments of the present invention, measurement of the amount of magnetizable or magnetic objects attached to the sensor surface may be performed before the magnetizable or magnetic objects are withdrawn from the sensor surface or after the magnetizable or magnetic objects are withdrawn from the sensor surface.

Measurement or detection of magnetic or magentizable objects attached to the senosr surface can be performed with or without scanning of the sensor element with respect to the biosensor surface.

According to preferred embodiments, measuring the amount of magnetizable or magnetic objects attached to the sensor surface may be performed more than once and the method may furthermore comprise:

monitoring the amount of attached magnetizable or magnetic objects as a function of time.

The magnetizable or magnetic objects may be coated with a first capture moiety and the sensor surface may be coated with a second capture moiety, the first and second capture moiety being different from each other.

The first capture moiety may most preferably be compatible with a first part of the biological molecule and the second capture moiety may most preferably be compatible with a second part of the biological molecule, the first part being different from the second part.

Measuring the amount of attached magnetizable of magnetic objects to the sensor surface may be performed by any suitable detection method e.g. magnetic detection, optical detection, sonic detection or electrical detection. Preferably, the magnetizable or magnetic objects used as labels may be detected or, in other words, the amount of magnetizable or magnetic objects attached to the sensor surface may be measured optically, e.g. using optical imaging or optical scanning, preferably with an optical technique that is surface-sensitive, e.g. with a confocal scanning optical pick-up unit that detects the labels through an optically transparent substrate (similar to optical data storage techniques for example) or with an evanescent-field optical detection technique.

Subsequently attracting and withdrawing the magnetizable or magnetic objects to and from the sensor surface may be performed by means of magnetic forces. The magnetic forces may be generated on-chip or off-chip.

According to preferred embodiments of the invention, the enzymatic process may be an RNA or DNA nucleic acid amplification process.

Embodiments of the invention relate to methods and tools for amplifying nucleic acids i.e. RNA or DNA and determining the amount of amplified RNA or DNA or of a reagent involved in the amplification process. In this method the step of determining the amount of the amplified nucleic acid or of a reagent involved in the amplification process may be performed via magnetic detection, optical detection, sonic detection or electrical detection, and may be performed at least one time during the amplification process of said RNA or DNA. In the method of the present invention, the step of determining the amount of the amplified RNA or DNA or of a reagent involved in the amplification process may be performed via a method wherein the amplified nucleic acid or the reagent are attached to a sensor surface via one or more biological molecule(s) or capture molecules.

Particular embodiments of the present invention relate to methods and tools for performing such methods as described above wherein the one or more biological molecules which is/are used to bind the amplified nucleic acid is DNA, Peptide Nucleic Acid (PNA) or RNA which specifically binds the amplified nucleic acid or amplicon. Alternatively, the biological molecule can be a protein, a vitamin, lipid or carbohydrate or can be a combination of both a nucleic acid and a protein which ensures binding of the amplicon to the sensor surface.

Particular embodiments of the present invention relate to methods and tools for performing such methods wherein the nucleic acid which is amplified by an isothermal method or by a thermocycling method.

Further particular embodiments of the present invention relate to methods and tools for performing such methods wherein the primers which are used for the amplification (amplimer) are labelled with a magnetic particle. Alternative embodiments of the invention relate to methods and tools for performing such methods wherein the amplicon is detected by way of a specific hybridisation probe, which is labelled (in this embodiment the amplimers are not labelled with a magnetic particle).

The present invention further provides a system for monitoring an enzymatic process on a biological molecule. The system comprises:

a reaction chamber for allowing an enzymatic process to take place, the reaction chamber being for use with or provided with magnetizable or magnetic objects,

a sensor device having a sensor surface,

means for, during the enzymatic process, subsequently attracting and withdrawing the magnetizable or magnetic objects to and from the sensor surface, and

means for, at least once, after attracting the magnetizable or magnetic objects to the sensor surface, measuring the amount of magnetizable or magnetic objects attached to the sensor surface.

The means for measuring the amount of magnetizable or magnetic objects attached to the surface may be such that measurement of the amount of magnetizable or magnetic objects attached to the sensor surface may be performed before the magnetizable or magnetic objects are withdrawn from the sensor surface or after the magnetizable or magnetic objects are withdrawn from the sensor surface

The means for measuring the amount of magnetizable or magnetic objects attached to the surface may be such that measurement or detection of magnetic or magentizable objects attached to the sensor surface can be performed with or without scanning of the sensor element with respect to the biosensor surface.

According to embodiments of the invention, the system may furthermore comprise means for monitoring the amount of attached magnetizable or magnetic objects to the sensor surface as a function of time.

The methods and tools of the present invention allow, for example, polynucleotide determination with improved analytical performance, such as improved speed, sensitivity and specificity. The methods and tools of the present invention allow polynucleotide concentration determination with improved ease of use, such as a higher robustness, a lower error rate, simpler interconnections and lower cost.

In one aspect of the present invention and in order to measure a concentration of, for example, nucleic-acids in a sample, magnetic-particle cycling is combined with nucleic-acid amplification, taking advantage of the integration and synchronisation of the magnetic cycling and the nucleic-acid amplification cycling processes (e.g. temperature cycling or reagent cycling).

The methods according to embodiments of the present invention may include the following steps: an optional nucleic acid extraction step, an optional magnetic pre-amplification step to determine the amount of starting nucleic acid, a nucleic acid amplification step and a detection step wherein a biomolecule (e.g. DNA or RNA) is used for binding the amplified nucleic acid, e.g. to a sensor surface or to another body.

The methods of the invention involve the detection of the amplified nucleic acids or of reagents involved in the amplification process by binding to a biomolecule which is itself bound to a sensor. The binding of the biomolecule to the sensor surface can be covalent.

According to one embodiment this process may comprise the following steps.

Amplification of nucleic acids in a bulk solution with primers covalently attached to magnetic particles and/or a sensor-chip surface.

Measurement of nanoparticles in the vicinity of a magnetic-label sensor (e.g. bound via a biomolecule to the sensor surface) as a function of time, and

Application of magnetic-particle cycling to allow the first bulk amplification process and the second surface detection process to take place with high efficiency.

More specifically, when using the PCR method, the method of the invention is characterized by the application of temperature cycling and magnetic force actuation to enable detection of amplification progress in near-real-time mode.

Generally, the present invention provides methods that combine amplification of nucleic acids with sensitive magnetizable or magnetic object detection. The system is advantageous in terms of real-time detection, speed, process control, process monitoring, multiplexing, compactness, ease of use and low cost.

The method and system according to embodiments of the invention are suited for sensor multiplexing (i.e. the parallel use of different sensors and sensor surfaces), label multiplexing (i.e. the parallel use of different types of labels) and chamber multiplexing (i.e. the parallel use of different reaction chambers).

The method and system according to embodiments of the present invention can be used as rapid, robust, and easy to use point-of-care biosensors for small sample volumes. The reaction chamber can be a disposable item to be used with a compact reader and may containing one or more magnetic field generating means and one or more detection means. Also, the method and system according to embodiments of the present invention can be used in automated high-throughput testing. In this case, the reaction chamber is e.g. a well plate or cuvette, fitting into an automated instrument.

### BRIEF DESCRIPTION OF THE DRAWINGS

FIG. 1 shows alternative configurations of magnetic-particle sensors for detection of amplified nucleic acid sequences for near-real-time detection in accordance with embodiments of the present invention.

FIG. 2 shows an example of a magnetic sensor for detection of amplified nucleic acid sequences in a two-step configuration with modules for amplification and free primer purification followed by the magnetic-particle sensor chip detection in accordance with embodiments of the present invention. After detection, the amplified nucleic acid is re-introduced into the amplification module.

FIG. 3 shows an example of a magnetic-particle sensor for detection of amplified nucleic acid sequences in a real-time configuration with a single, three chamber module for amplification, purification and detection (after detection, the amplified nucleic acid re-introduced into the amplification module.) (A), or with a single, one chamber module for amplification and detection (B) in accordance with embodiments of the present invention.

FIG. 4 shows an illustration of a device with a reaction chamber and magnetic nanoparticles (inlet and outlet are not shown) in accordance with embodiments of the present invention. The particles are actuated to go through a magnetic-particle cycling process, synchronised with the biochemical amplification process.

FIG. 5 shows a graph of sensor signal as a function of time.

FIG. 6 shows a schematic method in accordance with an embodiment of the present invention. PCR amplification is performed in the incubation chamber above the sensor chip. Each cycle, a particular combination of temperature and magnetic field actuation is applied to measure the near-real-time status of the amplification process.

### DETAILED DESCRIPTION OF THE EMBODIMENT

Amplimer refers to a nucleotide such as a DNA or RNA oligonucleotide used as primer for a DNA or RNA polymerase. The amplimers in a PCR reaction are generally called forward and reverse primers. A labelled amplimer refers to an amplimer which is covalently linked to a magnetic microparticle, or nanoparticle and/or a biological molecule, of which non limiting examples are biotin and fluorescein.

Amplicon refers to a nucleic acid obtained as a result of an amplification process.

Hybridisation probe or hybridisation primer refers to a nucleotide, such as a DNA or RNA oligonucleotide, which is used to detect amplified DNA or RNA (i.e. amplicon). In certain embodiments of the present invention a hybridisation probe can be covalently linked to a magnetic microparticle or nanoparticle and/or a biological molecule.

Sensor probe or sensor primer refers to a nucleotide such as a DNA or RNA oligonucleotide, which is directly or indirectly bound to the sensor surface (i.e. the part of the device which is the in proximity of or in the focus of or in the region-of-detection of a magnetic-particle detection system) and is capable of binding the amplicon. In amplification techniques wherein the amplicon comprises RNA, an RNA sensor probe can be used to bind the amplified RNA. The binding of the sensor probe to the sensor surface can be covalent. Alternatively, the sensor probe can be linked to the sensor surface through one or more biomolecules (ligands, antibodies) covalently linked to a biological molecule. It should be noted that the sensor probe can be any probe that can form a specific biological bond with a biomolecule in solution, e.g. antibody, carbohydrate, . . . .

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

The present invention provides a method for monitoring an enzymatic process of a biological molecule, such as e.g. a nucleic acid amplification process or another conversion process, with magnetizable or magnetic object, e.g. magnetic particles, as labels, while detecting the binding/unbinding of the labels with respect to a sensor surface. This may be done by any suitable technique to detect the presence of magnetizable or magnetic particles on or near a sensor surface, based on any property of the magnetizable or magnetic objects, e.g. magnetic particles. The method according to embodiments of the invention is suited for sensor multiplexing (i.e. the parallel use of different sensors and sensor surfaces), label multiplexing (i.e. the parallel use of different types of labels) and chamber multiplexing (i.e. the parallel use of different reaction chambers). Furthermore, the method according to embodiments of the present invention can be used as rapid, robust, and easy to use point-of-care biosensors for small sample volumes. The reaction chamber can be a disposable item to be used with a compact reader and may containing one or more magnetic field generating means and one or more detection means. Also, the method and system according to embodiments of the present invention can be used in automated high-throughput testing. In this case, the reaction chamber is e.g. a well plate or cuvette, fitting into an automated instrument.

Any chemical reaction or series of reactions catalysed by at least one enzyme is described as an enzymic or enzymatic process. Enzymes comprise a large group of proteins that increase reaction rates of biochemical processes and are produced by all living cells, and act both inside and outside of cells.

A lot of different enzymes exist. Most naturally occurring enzymes are highly specific, meaning that each enzyme usually catalyses just one defined biochemical reaction. In, for example, molecular biology, certain enzymes are tools for cleaving and rejoining DNA, the carrier of genetic information. “Restriction enzymes” recognise defined sequences of nucleotides to cut the DNA precisely at that site. Other enzymes (ligases) can link separate strands of DNA into one, continuous piece. The present invention is not limited to naturally occurring enzymes but also includes within its scope the use of semi-synthetic or synthetic enzymes. Semi-synthetic enzymes can bear non-natural functional groups including the replacement of the native cofactors by modified cofactors chemically linked to functional groups providing novel properties. Such enzymes have synthetic or artificial groups placed at specific positions in close vicinity to the cofactor site. However synthetic enzymes do not need to mimic natural enzymes in any way. Synthetic enzymes are disclosed for example in U.S. Pat. No. 5,110,833.

A biochemical enzymatic process can involve several molecular transformations, or in other words can be performed to any biological molecule. One type of an enzymatic process may, for example, be a nucleic acid amplification process.

The method according to the present invention comprises:

allowing an enzymatic process to take place in a reaction chamber, the reaction chamber being provided with magnetizable or magnetic objects,

providing a sensor device having a sensor surface,

during the enzymatic process, subsequently attracting and withdrawing the magnetizable or magnetic objects to and from the sensor surface, and

at least once during the enzymatic process, after attracting the magnetizable or magnetic objects to the sensor surface, measuring the amount of magnetizable or magnetic objects attached to the sensor surface.

Measurement or detection of the amount of magnetizable or magnetic objects attached to the sensor surface may be performed before the magnetizable or magnetic objects are withdrawn from the sensor surface or after the magnetizable or magnetic objects are withdrawn from the sensor surface.

Measurement or detection of magnetic or magentizable objects attached to the senosr surface can be performed with or without scanning of the sensor element with respect to the biosensor surface.

The present invention will further be described by means of a sensor device based on magneto-resistive elements. However, this is not limiting the invention in any way. The present invention may be applied to sensor devices comprising any sensor element suitable for detecting the magnetizable or magnetic objects, e.g. magnetic particles, on or near a sensor surface based on any property of the particles. For example, detection of the magnetizable or magnetic objects, e.g. magnetic particles, may be done by means of magnetic methods (e.g. magnetoresistive sensor elements, hall sensors, coils), optical methods (e.g. imaging fluorescence, chemiluminescence, absorption, scattering, surface plasmon resonance, Raman, . . . ), sonic detection (e.g. surface acoustic wave, bulk acoustic wave, cantilever, quartz crystal, . . . ), electrical detection (e.g. conduction, impedance, amperometric, redox cycling), . . . .

Preferably, the magnetizable or magnetic objects used as labels may be detected or, in other words, the amount of magnetizable or magnetic objects attached to the sensor surface may be measured, optically, e.g. using optical imaging or optical scanning, e.g. with a confocal scanning optical pick-up unit that detects the labels through an optically transparent substrate (similar to DVD for example).

Furthermore, the present invention will be described by means of the magnetizable or magnetic objects used as labels being magnetic particles. Again, this is only for the ease of explanation and does not limit the invention in any way. The present invention also applies for a magnetizable or magnetic object being a magnetic rod, a string of magnetic particles, or a composite particle, e.g. a particle containing magnetic as well as optically-active material, or magnetic material inside a non-magnetic matrix.

The present invention will further be described by means of the enzymatic process being an RNA or DNA nucleic acid amplification process. It has to be understood that this is only an example and is not intended to limit the invention in any way. The method according to the present invention may also be applied to other enzymatic processes.

Hence, in a first step, the method according to the present invention comprises allowing the RNA or DNA nucleic acid amplification process to take place in a reaction chamber. The reaction chamber comprises the sample with the RNA or DNA nucleic acids and furthermore comprises magnetic particles as labels. The magnetic particles may be coated with a first capture moiety. The first capture moiety may be any suitable capture moiety known by a person skilled in the art. Preferably, the first capture moiety may be compatible with a first part of the amplified RNA or DNA nucleic acid or amplicon, so that coated magnetic particles can attach to the RNA or DNA nucleic acid through the first capture moiety. For example, in the example given for RNA or DNA nucleic acid amplification the first capture moiety may be a first oligonucleotide.

Next, a sensor device having a sensor surface may be provided. The sensor surface may be coated with a second capture moiety which is most preferably different from the first moiety. The second capture moiety may preferably be compatible with a second part of the amplified RNA or DNA nucleic acid or amplicon different from the first part, so that the RNA or DNA nucleic acid can attach to the sensor surface. For example, in the example given for the RNA or DNA nucleic acid amplification process, the second capture moiety may be a second oligonucleotide, preferably different from the first oligonucleotide. The amount of magnetic particles that is attached to the sensor surface is a measure of amplified RNA or DNA nucleic acids or amplicons.

During the amplification process, the RNA or DNA nucleic acids attached to magnetic particles will subsequently being attracted to and withdrawn from the sensor surface. This may be done by means of magnetic forces. According to embodiments of the invention, the magnetic forces may be generated on-chip, e.g. by means of a magnetic field generating means, e.g. current wire, integrated in the sensor device. According to other embodiments of the invention, the magnetic forces may also be generated off-chip or externally, for example by means of an external magnet. During the amplification process, more and more amplicons or amplified RNA or DNA nucleic acids, will be generated over time. Hence, as the amplification process proceeds or in other words, as more amplification cycles are performed, more and more amplicons will attach to the magnetic particles. In other words, during the amplification process, more and more amplicons will, during the attract step, be attached to the sensor surface over time.

According to the method of embodiments of the present invention, the amount of magnetic particles attached to the sensor surface may be measured at least once. The measurement may be done after attracting the magnetic particles to the sensor surface. Preferably, the amount of magnetic particles attached to the sensor surface may be measured more than once during the amplification process. In this case it is possible to monitor the amount of attached magnetic particles to the sensor surface as a function of time.

Hereinafter some embodiments will be described for possible implementation of the amplification process and the detection, i.e. qualitative and quantitative determination, of the amplified nucleic acids.

In one aspect the invention relates to methods wherein nucleic acid amplification is alternated with the qualitative and quantitative determination of the amplified DNA using magnetic detection and tools for performing such methods.

Methods of the present invention include the following steps:

a nucleic acid amplification process (stepwise or continuous),

one or more detection steps during and/or after the amplification process wherein a DNA or RNA sensor probe is used for binding the amplified nucleic acid, e.g. to a sensor surface or to another body. The binding of the sensor probe to the sensor surface can be covalent.

Optionally, the methods of the invention further comprise, prior to the amplification:

a nucleic acid extraction step, and/or

a magnetic pre-amplification step to determine the amount of starting nucleic acid.

According to one embodiment, the detection of the amplified nucleic acid or amplicon is ensured by incorporating the label into the amplicon. According to this embodiment the method of the invention comprises the following steps:

amplifying nucleic acids in a bulk solution with primers attached to magnetic nanoparticles or microparticles

contacting the amplified nucleic acid with a sensor probe attached to sensor-surface. The attachment of the primers to a sensor surface may be covalent.

Detection or measurement of the magnetic nanoparticles or microparticles at least once during and/or after the amplification process.

Optionally, magnetic-particle cycling is applied to allow the (first) bulk amplification process and (second) surface detection processes to take place with high efficiency.

In a specific embodiment of the invention, temperature cycling and magnetic force actuation are applied to enable detection of the amplification progress in near-real-time mode.

As indicated above, the methods of the present invention include a nucleic acid amplification step, e.g. in bulk liquid. Different amplification methods which are known in the art can be integrated in the methods of the present invention. Amplification protocols are commonly divided into target and probe types of amplification (Hill, C. S. (1996) Journal of Clinical Ligand Assay 19, 43-52.).

‘Target’ amplification yields copies of the desired target sequence by synthesis from individual nucleotides using the target nucleic acid molecule as template. Examples are polymerase chain reaction (PCR), transcription mediated amplification (TMA), nucleic acid based sequence amplification (NASBA) and strand displacement amplification (SDA) (Saiki et al. (1988) Science 239, 487-491; Compton, J. (1991) Nature 350, 91-92; Walker et al. (1992) Nucl. Acids Res. 20, 1691-1696; McDonough et al. (1997) In: Nucleic acid amplification technologies, Eds. Lee, H., Morse, S. and Olsvik, O., Natick, M A: Biotechniques Books, 113-123.). Specific embodiments of the invention involve the amplification of a specific DNA or RNA using PCR. On the other hand ‘probe types’ of amplification produce modified versions of the original probes put into the reaction. An example of this approach is the ligase chain reaction (LCR) (Laffler et al. (1993) Annales de Biologie Clinique 50, 821-826).

In the case of PCR and LCR a thermocycler is used to denature double-stranded intermediates. Other protocols (e.g. TMA, NASBA and SDA) are isothermal and require generally only one heating device at constant temperature (e.g. water bath or thermoblock).

Transcription methods such as TMA have several differences compared to PCR/LCR. TMA can use either RNA or single-stranded DNA directly as a target. Theoretically, these methods are faster than PCR/LCR in that they can produce a billion-fold amplification in as little as 15 minutes, while PCR/LCR can take 3-4 hours to produce a similar amount. On the other hand TMA is potentially less specific than PCR, because the process is performed at a lower temperature. Specific probes are used to compensate for this difference.

A recent technique (EXPAR) uses a combination of a heat stable nicking enzyme and a polymerase (Van Ness et al. (2003) Proc. Natl. Acad. Sci. 100, 4504-4509). It is an isothermal molecular chain reaction in which short oligonucleotides are generated. The method is highly sensitive and can achieve amplifications of >10⁶-fold. The robustness, speed, and sensitivity of the exponential reaction is useful in rapidly detecting the presence of small amounts of a specific DNA sequence in a sample.

The different amplification techniques above are generally referred to as amplification processes, i.e. a process from the beginning of the amplification of a sample until the desired amount of amplicon is obtained, or until the amplimers are exhausted and/or the enzyme(s) involved in the amplification have lost their activity.

In the case of thermocycling amplification process, this process comprises as such a number of discrete amplification steps. In the case of isothermal processes, the amplification is a continuous process. By magnetic cycling with probe-labelled particles and another probe immobilized onto the sensor surface the amplified material (e.g. a single-stranded RNA oligonucleotide in the case of NASBA) can be bound to the surface to enable detection of near-real-time qualitative or quantitative data. Preferably, the probes hybridise to a sequence of the amplified material different from the amplimer sequences.

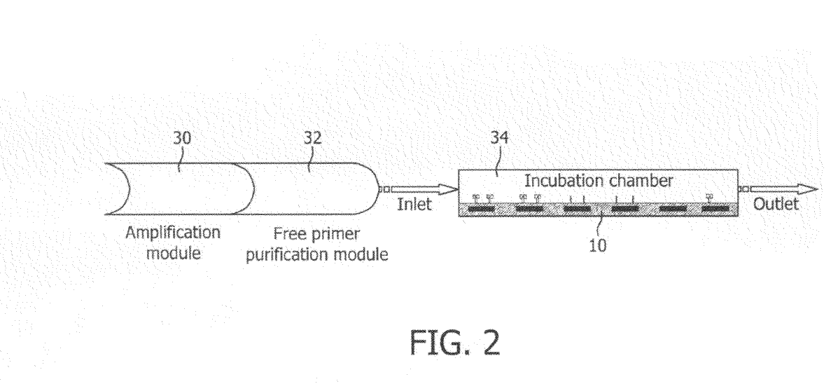

Alternatively, this process can be however divided into amplification steps by manipulating the temperature or by physically separating the template from the amplimers. When referring to the method of the present invention as a two-step method, a method whereby amplification (and optionally purification) and detection are performed in different units, modules or chambers of the device used. Typically, the two-step method of the invention will be performed in a device comprising an amplification module, in which amplification is performed and an incubation chamber comprising the sensor (chip) surface (FIG. 2). Optionally, the device comprises a third module wherein the free primers are purified.

According to a particular embodiment of the invention a sample is pre-treated, prior to the (first) amplification, with an RNA and/or DNA extraction step. Suitable protocols are described in reference manuals on molecular cloning (e.g. Sambrook et al. 1989). Several types of DNA or RNA extraction kits are commercially available (Pharmacia, Dynal, Waters). These extraction methods (typically making use of magnetic particles) remove disturbing compounds such as polysaccharides and polyphenols.

Also simple extraction methods can be used wherein rRNA is extracted which is present in thousands of copies in a single cell (Kohne et al. 1984) In Thornsberry et al. (Ed): Legionella: Proceedings of the 2nd International Symposium, Washington D.C., American Society for Microbiology, 107-108). The use of a certain pre-treatment depends on parameters such as complexity and concentration of the sample.

In a further optional step, the nucleic acid, which is initially extracted using magnetic particles, can be directly used for detection by a magnetic sensor chip, in order to determine the amount of starting material in a pre-amplification step.

According to embodiments of the invention, the methods of the present invention may comprise a detection step using magnetic sensors. According to a specific embodiment of the method of the invention, a biomolecule is used for binding the amplified nucleic acid to a sensor surface. The detection step is performed at least once during the amplification process, and also optionally at the end of the amplification process. In continuous amplification processes the detection can be performed at certain time points during the amplification process. In stepwise amplification processes such as PCR the detection step is typically performed after the extension step. The detection step can be performed after each amplification cycle of the amplification process. In certain embodiments wherein a quantitative determination is desired, the detection step takes place in the exponential phase stage of a PCR reaction, typically between about cycle 15 to about cycle 25.

The biomolecule or first capture moiety, used for binding the amplicon to the sensor or to a molecule allowing detection by said sensor can be a protein, a nucleic acid but also other biological compounds such as carbohydrates or vitamins (e.g. biotin).

According to a particular embodiment of the invention the amplicon is linked directly or indirectly to the sensor surface by way of a sensor probe or second capture moiety which is an oligonucleotide specific for the amplicon. Optionally, this sensor probe is covalently linked to the sensor surface.

All types of the herein-defined probes, including the sensor probe or hybridisation probe, can also be linked to other molecules such as proteins, or organic molecules, either directly to the oligonucleotide, or via attachment to a magnetic particle, which is bound to the oligonucleotide. For certain applications cleavable linkers are envisaged (e.g. chemical linkers cleavable by a reducing agent, or proteins or DNA fragments which can be enzymatically cleaved). Biological interactions which ensure binding between the biological molecules envisaged in the present invention are for example DNA/DNA binding, DNA/RNA binding, antigen-antibody binding, ligand-receptor binding, substrate-enzyme binding, inhibitor-enzyme binding, affinity binding (e.g. biotin-(strept)avidine, Zinc-His-Tag, GST-GST binding protein, etc.)

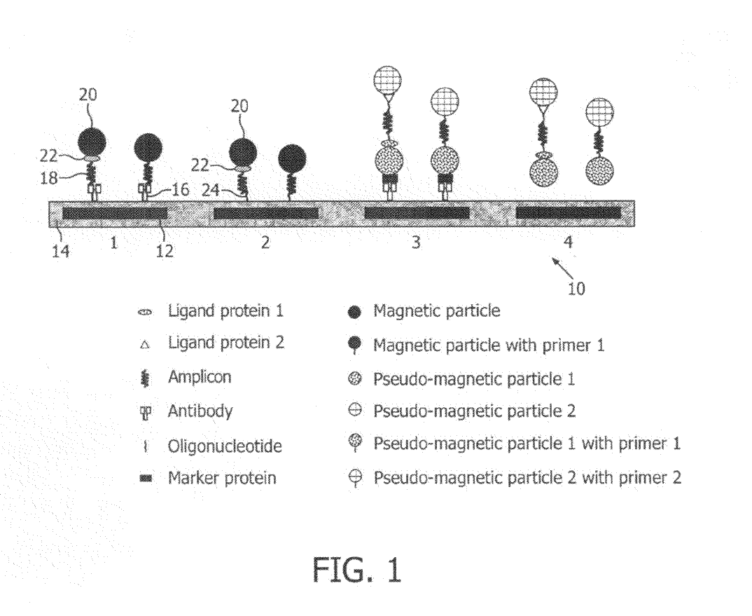

According to one embodiment, biological molecules acting as capture molecules are immobilized on the surface of the sensor such as e.g. antibodies, a special attractive protein or polymer surface, etc. These capture molecules specifically bind the amplicons, e.g. through a tag bound to a primer (sensor probe). FIG. 1, shows in item 1 various capture schemes that can be used with the present invention. For example a capture molecule 16 such as an antibody may be bound to the surface of a sensor 10 which comprises magnetic field generators 12, e.g. electrical current wires embedded in a substrate 14. The capture molecule 16 may be covalently bonded to the sensor surface. The capture molecules 16 are bound to the sensor surface at least in the vicinity of the magnetic field generators 12. The substrate 14 can be an organic or inorganic substrate, e.g. a polymer, semiconductor or glass substrate, e.g. of a “biochip”. The antibody 16 binds specifically to an amplicon 18 generated by the amplification step either directly or through a tag linked to a probe specific for the amplicon. A magnetic or magnetizable particle 20 is bound to the amplicon 18 through a ligand protein 22 or by another means, e.g. directly (incorporation during amplification) or through a hybridization probe.

In an alternative embodiment of the present invention, the sensor-probes are linked directly to the sensor surface, this is the sensor-immobilised ligands are in fact specific oligonucleotide sequences 24 that hybridise to the end of the amplicon 18 which is not attached to the magnetic particle 20 as shown in FIG. 1, item 2. A magnetic or magnetizable particle 20 is bound to the amplicon 18 through a ligand protein 22 or by another means, e.g. directly (incorporation during amplification) or through a hybridization probe.

Magnetic or magnetizable particles 20, used in the present invention are typically in the range of 10 nm to 5 micrometer, i.e. magnetic or magnetizable nanoparticles or microparticles. The size of the magnetic or magnetizable particles 20 should not be too small, to facilitate detection and to be able to actuate the particles with applied magnetic fields. The size should also not be too large, because this can lead to non-specific binding, and sedimentation. Preferably, the particle size is in the range between 30 nm and 3 micrometer, more preferred between 60 nm and 1 micrometer. The particles structure and shape can be any suitable one, e.g. single-magnetic-core, multi-magnetic-core, spherical, rod-shaped, etc. Magnetic or magnetizable particles 20 can be further coated with (bio)polymers to enhance stability and to provide functional groups for attaching a biological molecule as mentioned above. Also, components can be added to facilitate magnetic-particle detection, e.g. luminescent material in the case of optical detection.

A sensor 10 to be used in accordance with the present invention detects or measures the presence of magnetic micro-particles or nanoparticles 20 in the vicinity, for example within 100 μm, more preferred within 10 μm. According to a particular embodiment, the sensor 10 will be ten times more sensitive to nanoparticles within 1 μm of the sensor surface than to nanoparticles at a distance of 10 μm from the sensor surface. The sensor 10 can be any suitable sensor that can detect the presence of magnetic particles (see earlier). Preferably the sensor 10 is an optical-based or a magnetic-based sensor that is sensitive to magnetic labels attached to the sensor surface. The sensor can optionally be integrated in a chip.

On the sensor surface or in the proximity of the sensor surface preferably as many functions as possible are integrated, such as temperature sensing, heating, and magnetic-particle detection. Cooling elements (e.g. Peltier element) can be integrated in the cartridge or can be part of the reader instrument. When rapid heating and cooling is desirable (e.g. PCR) the contact area of the sample liquid to the heater or cooler element is preferably as large as possible.

The strength of field and field gradient required to transfer magnetic particles through a solution depend on several parameters, e.g. the magnetic susceptibility and magnetic moment of the particles, the homogeneity and concentration of particles, the occurrence of particle-particle interactions such as clustering or chain formation, and the flow resistance in the medium. The fields can be generated by a combination of field-generating means (current wires and magnetic material) in the reader, in the cartridge, and on the chip. In our system these parameters will be selected in such a way to give repetitive transport of particles in the biological chamber during the time of the assay.

Biological bonds between a magnetic particle and another element (e.g. the sensor surface) can be probed or disrupted by generating forces or torques between the particle and the other element (see for example WO2005010527).

Magnetically labelled biomolecules are introduced (e.g. labelled primers and probes) and generated (e.g. the labelled amplicon) in the reaction mixture and are then manipulated. In particular embodiments, the magnetically labelled biomolecules are distributed and/or mixed within this mixture by applying magnetic fields. For example, magnetic-particle surface/bulk cycling in biochemical assays are used as has recently been described in an immunoassay with particle detection by a planar coil (Luxton et al. (2004) Anal. Chem. 76, 1715.).

The present invention includes additional methods for magnetic-particle cycling on magneto-resistive sensors and methods to make the bulk as well as the surface processes more efficient, e.g. using additional agents or dedicated actuation methods as described in WO2005010527.

Biological interactions with microparticles or nanoparticles can give a strong increase of the steepness of a melting curve and enhanced specificity of detection, possibly due to co-operative effects.

Different devices and substrates are suitable for performing the methods of the present invention. The device can have a flow-over or a flow-through design. According to embodiments of the invention, the substrate can be a flat substrate, a substrate with surface structure or a porous substrate. A flow-through package is for example described in DE040286_EPP (filed in November 2004). Care is taken to avoid dead corners or unwanted re-circulation, e.g. having smooth transitions and by avoiding flow edges with steep angles. In case a washing step is used with a washing solution, the design of the chamber preferably ensures a large refresh rate over the sensor surface, e.g. by narrowing the flow depth at the location of the sensor. The cartridge is preferably made of materials that have low non-specific binding of biomaterials, in order to avoid the loss of target material and/or reagents to the cartridge walls.

FIG. 2 illustrates one embodiment of the invention using a serial arrangement of amplification 30, purification 32 and detection 34 units. The amplification process is separated from the sensor detection step. Undesired interactions between free primers and specific ligands at the sensor surface are avoided and if necessary, free primers and other reaction contaminants remaining after the amplification process are removed in a purification module 32, consisting of, for example, silica material. According to certain embodiments the fluid in the amplification chamber is recirculated, whereby the purification module is designed to allow the amplicon to pass and maintain all other materials (e.g. the free primers) in the amplification chamber.

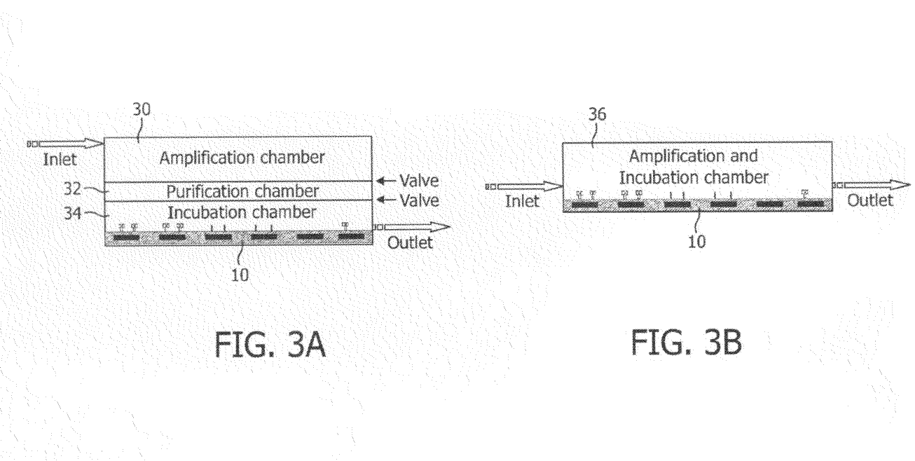

FIG. 3A illustrates one embodiment of the invention whereby a single device is used with three chambers 30, 32, 34, each transition separated by a controllable valve-system to allow the controlled and timely passage of the solution into the next chamber. The three chambers 30-34 and sensor chip 10 fulfil similar roles as the modules 30-34 and sensor chip 10 in the construction as depicted in FIG. 2, but here the fluids can be transferred back-and-forth between the chambers 30-34.

In a further preferred embodiment a processing and detection device is as shown in FIG. 3B, i.e., amplification and detection or measurement are performed in a single device 36, preferably in a single chamber in the vicinity of the sensor chip 10, which allows direct detection of amplicons formed. More particularly the device comprises a heat stable sensor surface, preferably with immobilised ligands linked thereto and has the ability to perform the amplification protocol at varying temperatures (e.g. PCR), or at a constant temperature (e.g. NASBA) as required by the amplification method used. Additionally, the device is adapted to allow the removal of competitive free primers if present, to avoid the occurrence of non-specific interactions such as in primer-primer complexes and to take account of the specific characteristics of magnetic particles used.

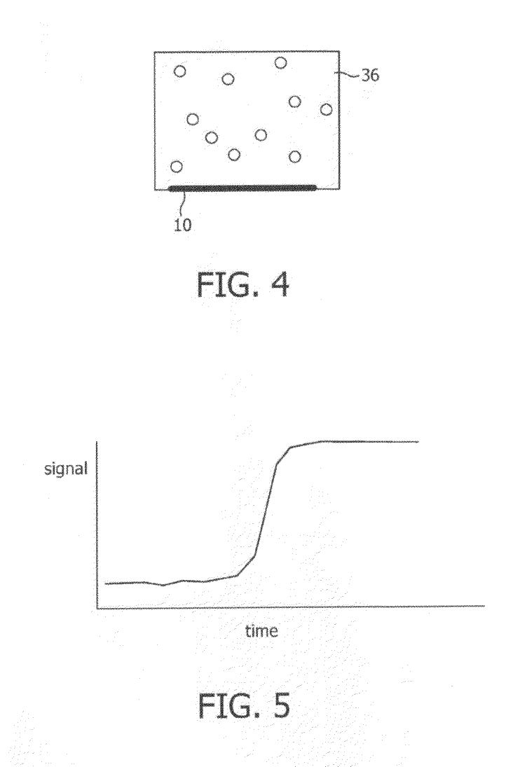

As shown schematically in FIG. 4, the present invention includes additional methods for magnetic-particle cycling on magnetic-particle sensors and methods to make the bulk as well as the surface processes more efficient, e.g. using additional agents or dedicated actuation methods as described in WO2005010527.

Modifications of the processing and detection devices are included within the scope of the present invention. In particular, other possible device geometries include the use of high-surface-area or porous materials for the sensor surface. Further a lateral flow or flow-through architecture can be used.

The present invention is applicable in a variety of applications. A non-limiting list of applications are for example, the detection of micro-organisms (food spoilage and poisoning, and of genes encoding toxins in the agri-food-environment field; the assessment of active genes (mRNA in ‘genomics’ studies) in crops and fruits (quality indicators); the detection of GMO's (food safety and identification) the assessment of adulteration/fraud (determination of foreign DNA); the detection of allergenic products/contaminations; the detection of micro-organisms with environmental consequences, e.g. MRSA, legionella, listeria; the identification of micro-organisms in cell cultures. Applications in clinical diagnostics are for example: the detection of micro-organisms with respect to sepsis, meningitis, respiratory diseases, tuberculosis, hepatitis, AIDS, etc.; the identification of micro-organisms in cell cultures, e.g. related to the above diseases.

The invention is now illustrated with the following examples.

### Example 1

### PCR Amplification using Amplimers Without Magnetic Label

After an optional nucleic acid extraction step, the amplification of nucleic acids is performed in any of the processing and detection devices mentioned above according to the PCR protocol with two specific amplimers in the solution. The amplification process results in amplicons 18 of which one of the strands hybridises with a hybridisation probe which is covalently bound to the surface of magnetic nano- or microparticles 20. Another part of this strand of the amplicon 18 hybridises with another complementary probe, i.e. a sensor probe, attached to the sensor chip surface. The attachment may be covalent. Minimal interference is obtained when the hybridisation and sensor probe hybridise with a nucleic acid sequence within the extended strand that is not complementary to the amplimers used in the amplification process. In this example, the concentration of microparticles or nanoparticles which become attached to the sensor is intermittently measured, at a particular point in a repeating sequence (cycle).

The three following steps describe a conventional PCR cycle:

Template double-strand DNA is heated to dissociate the individual strands, typically between 90 and 99° C. (denaturation).

The temperature is lowered to allow primers to anneal to the free strands, typically between 45-65 depending on the amplimer primer length and sequence (annealing).

The temperature is raised, typically to 72° C. Primers are extended along the template strands (extension).

In order to detect and measure the amplified nucleic acid, e.g. DNA, RNA the following steps are performed:

After completion of the DNA amplification, the temperature is raised to dissociate double-strand DNA.

Hybridisation probes with magnetic micro- or nanoparticles are attracted from the bulk solution toward the sensor surface using a magnetic force, e.g. due to a magnetic field gradient generated by integrated magnetic field generators 12 and/or by magnetic-field generators external to the substrate. The DNA strand complementary to the hybridisation and the detection probes, is sandwiched (via hybridisation) between these probes. As a consequence the DNA strand is immobilised to the surface of the sensor and labelled with a magnetic particle.

Non-bound hybridisation probes are subsequently repelled from the sensor surface by applying a magnetic force, e.g. by a magnetic field and/or magnetic field gradient with the correct slope, however the maximum force being such that it does not disrupt the DNA-DNA hybrids in the strand-hybridisation probe-sensor probe complex. Typical values are in the pN range, forces in the nN range will disrupt the specific bonds (as described in WO2005010527).

The magnetic field strength of the magnetic particles attached to the hybridisation probe is measured, this value being related to the amount of amplicon formed in the PCR amplification. This method allows a near-real-time PCR detection. In its most commonly used form, real-time PCR detects a bulk nucleic-acid amplification process using molecular reporters (e.g. molecular beacons) that are dissolved in the bulk of the fluid. The state of the molecular optical reporters is detected using an optical detection system that scans or images the volume of the fluid. A disadvantage of volume-based detection techniques is that the detection limit is rather high. As a consequence, the nucleic-acid amplification process needs to run for more cycles in order to get a detectable signal. Surface-based detection techniques can in principle reach lower detection limits than volume-based techniques, but this requires that specific molecular materials are concentrated from a volume toward a surface, which is time-consuming due to slow diffusion processes. Therefore, it is advantageous to use magnetic labelling of molecular material, such that the specific material can be attracted toward the sensing surface.

After the measuring step, the temperature may optionally be raised again to dissociate probe amplicon strand interactions. The nanoparticles are redistributed in the bulk solution by inverting the magnetic field gradient whereafter they participate again in the biochemical reactions by magnetic force actuation applied. At this point the sample is denatured and ready to enter a next amplification cycle.

FIG. 5 shows a graph of sensor signal as a function of time.

For those skilled in the art it is evident that modifications of particular steps in the above process are possible. For example, the detection step is performed after each amplification step, alternatively the detection step is performed with a lower frequency or is only performed after an initial number of PCR cycles have been performed without detection step. Also, after each detection step measures can be taken to remove all attached magnetic particles; alternatively, the attached magnetic particles may remain on the surface and the non-attached particles may be redistributed in the bulk solution to participate again in the biochemical reactions.

The above process is called ‘magnetic particle and temperature cycling’ as both the magnetic particles are cycled as well as the temperature. It allows micro- or nanoparticles to efficiently participate in a bulk process as well as in a surface process. In this embodiment amplicon formation and generation takes place in the bulk solution and particle detection at the sensor surface. It is a form of intermittent detection, the time between individual measurements being equal to the cycle time of the PCR procedure. By decreasing the PCR cycling time, the near-real-time situation of the amplification process becomes more accurate. Presently, state-of-the-art, miniaturized amplification requires 15 to 30 seconds per cycle.

By addition of an internal standard to the process, i.e. a known amount of a reference template and dedicated primers for comparison purposes, it is possible to make this near-real-time amplification also quantitative. Preferably, a separate spot or spots (statistically randomised) on the sensor surface is or are made specific for this internal standard. By recording the amplification efficiency (e.g. amount per time interval) a calculation of the initial amount of the unknown template DNA is possible.

In a more preferred set up the detection is performed in a regime where the amount of hybridisation primers to the micro- or nanoparticles has a low amplicon coverage, i.e. on average less than one amplicon per nanoparticle. This ensures that the probability of having more than one amplicon per micro- or nanoparticle is very low and that the number of micro- or nanoparticles on the sensor can be quantitatively and accurately translated into a target concentration in the original sample.

In accordance with the present invention multiplexing may also be performed by having an array of sensors 10 with different capture molecules. In some cases the same primers can be used, in other cases different primers will be needed.

Besides sensor multiplexing, also label multiplexing can be important, e.g. for comparative assays, for the addition of controls and for further parallellization. Label multiplexing refers to the fact that different types of labels can be distinguished in an assay. An advantage of using particle-based labels is that label multiplexing is easily incorporated, because labels can be provided with different properties. An advantage of using nano- or microparticle labels with respect to molecular labels is that components can be added to or incorporated into the labels, which gives a lot of possibilities for label multiplexing. For example, in the case of optical detection label multiplexing can be achieved by using magnetic labels with different optical properties. Providing magnetic labels with different properties can be done by several ways known in the art, e.g. by adding dyes with different spectral characteristics, such as luminescent properties, refractive properties, absorption properties, Raman properties, scattering properties, phosphorescence properties, etc., to the magnetic labels.

### Example 2

### PCR Amplification using Amplimers with Magnetic Label

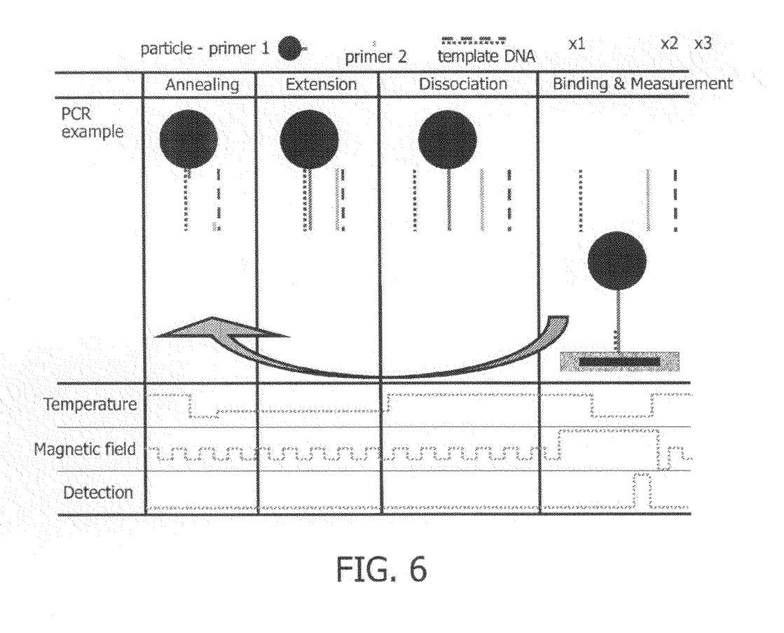

After an optional nucleic acid extraction step, amplification of nucleic acids is performed according to the PCR protocol with amplimers of which one is coupled covalently to a magnetic particle and the other is not magnetically labelled. This example is illustrated schematically in FIG. 6. The amplification process results in one of the extended strands being labelled to magnetic micro- or nanoparticles via the attached primer.

In this example, the concentration of micro- or nanoparticles attached to the sensor 10 is intermittently measured, at a particular point in a repeating sequence (cycle).

The steps describing a preferred design and procedure and the additional options indicated according to this embodiment are essentially the same as described for the procedure in Example 1, although apart from the amplimers only one additional probe (sensor probe) is necessary, attached covalently to the sensor chip surface. This sensor probe can comprise the same sequence as an amplimer oligonucleotide or can overlap with the sequence of an amplimer oligonucleotide, as depicted in FIG. 6. Alternatively, the sensor probe hybridises to a sequence of the amplified DNA different from the sequence to which an amplimer binds.

As an alternative of the above: the concentration of magnetic particles bound to the sensor during a detection step, has a relationship with the original target concentration. In other words, for a given assay time and time of the detection process, the concentration of particles on the sensor by binding to a formed amplicon strand indicates the original target concentration. Another way of measuring the amount of amplicon formed is the detection of the amount of amplimers remaining at a particular assay time. If the particle-bound amplimer is targeted for this approach, the concentration of bound particles would decrease upon increasing concentration of amplicons formed during the amplification process. Here an example of such an assay is given:

One type of amplimer is coupled covalently to a magnetic particle. On the sensor, probes are immobilized which have been selected to bind to this amplimer. So initially the particles with this covalently-coupled amplimer can bind to the sensor with a high binding rate. As the amplification process proceeds, more particle bound amplimers will be extended to full amplicons. As a consequence, the probability that a particle binds to the sensor decreases, driven by the lower number of accessible amplimers on the particle and/or due to steric hindrance by the amplicons bound to the particle.

### Example 3

### Microarray Applications of the Invention

The procedures described in the previous examples enable the detection of amplified genetic material by magnetic field cycling and in some cases temperature cycling. Any of these methods is excellently suited to be applied in micro-array applications. The strength of interaction between probes immobilised at the sensor surface of the array and amplicons from the sample solution (in this case attached to magnetic nanoparticles) is more or less proportional to the extent of complementarity between probe and amplicon. By applying different forces across the array (e.g. different magnetic forces that depend on the array position) or by varying the forces as a function of time, various types of array sub-spots are identified, e.g. from spots loosing the attached amplicons and magnetic nanoparticles at low forces (e.g. non-specific or highly-degenerative hybridisation) up to spots with a very powerful binding between probe and amplicon (e.g. high complementarity). In this last case the magnetic signal is still recordable as a consequence of the magnetic particles immobilised at these particular spots.

In a micro-array set up, in which specific probes are immobilized onto very small and discrete parts of the sensor surface, the chance of collisions between a particular probe and a labelled magnetic particle having the specific complementary amplicon/ligand attached or bound, is very small. Magnetic force cycling with velocity components perpendicular to the sensor surface will increase the concentration of specific binding pairs (i.e., surface probe and amplicon/ligand labelled magnetic particle) in a small volume above the sensor surface. However, movement of particles parallel to the sensor surface is restricted (also as a result of the magnetic field). Consequently, the interaction of specific binding pairs in relation to the amplification process necessary to give measurable results is an inefficient process in the micro-array set up.

To overcome this drawback another embodiment of the micro-array format is presented in which magnetic forces and velocities are applied with components parallel to the sensor surface, intermittently or in a co-ordinated way with the perpendicular components, to move the particles horizontally and in close proximity over the sensor surface to further increase collisional contacts.

In addition to adjusting the temperature to influence the binding efficiency magnetic field actuation can be used to give added value to sub-divide interactions at a particular temperature. This may enable the detection of SNPs or other changes of nucleic-acid sequence, so that gene-differences can be detected without sequence analysis (note that a sequence analysis may be used as a confirmation test). This may also enable a more sensitive and more accurate description of up- and down-regulated genes, i.e., a lower number of key-genes with respect to the particular physiological parameter studied in the micro-array experiment. This may also enable a more precise identification of micro-organisms, pathogens, or other biological material.

1. A method for monitoring an enzymatic process on a biological molecule, the method comprising:
allowing an enzymatic process to take place in a reaction chamber, the reaction chamber being provided with magnetizable or magnetic objects,
providing a sensor device having a surface,
during the enzymatic process, subsequently attracting and withdrawing the magnetizable or magnetic objects to and from the sensor surface, and
at least once during the enzymatic process and after attracting the magnetizable or magnetic objects to the sensor surface, measuring the amount of magnetizable or magnetic objects attached to the sensor surface.

2. A method according to claim 1, wherein measuring the amount of magnetizable or magnetic objects is performed more than once and wherein the method furthermore comprises:
monitoring the amount of attached magnetizable or magnetic objects to the sensor surface as a function of time.

3. A method according to claim 1, wherein the magnetizable or magnetic objects are coated with a first capture moiety and the sensor surface is coated with a second capture moiety, the first and second capture moiety being different from each other.

4. A method according to claim 3, wherein the first capture moiety is compatible with a first part of the biological molecule and the second capture moiety is compatible with a second part of the biological molecule, the first part being different from the second part.

5. A method according to claim 1, wherein measuring the amount of attached magnetizable of magnetic objects is performed by magnetic detection, optical detection, sonic detection or electrical detection.

6. A method according to claim 1, wherein subsequently attracting and withdrawing the magnetizable or magnetic objects to and from the sensor surface is performed by means of magnetic forces.

7. A method according to claim 1, wherein the enzymatic process is an RNA or DNA nucleic acid amplification process.

8. A method according to claim 7, wherein the amplification process comprises amplifying RNA or DNA nucleic acid using one or more amplimers and determining the amount of said amplified RNA or DNA nucleic acid or of a reagent involved in the amplification process.

9. The method according to claim 8, wherein the RNA or DNA is amplified by an isothermal method.

10. The method according to claim 8, wherein the RNA or DNA is amplified via a thermocycling method.

11. The method according to claim 8, wherein said amplified RNA or DNA is detected using a magnetically labelled hybridisation probe.

12. The method according to claim 1, further comprising magnetic force cycling with velocity components perpendicular to the sensor surface.

13. A system for monitoring an enzymatic process on a biological molecule, the system comprising:
a reaction chamber for allowing an enzymatic process to take place, the reaction chamber being for use with magnetizable or magnetic objects,
a sensor device having a surface,
means for, during the enzymatic process and when the magnetizable or magnetic objects are present, subsequently attracting and withdrawing the magnetizable or magnetic objects to and from the sensor surface, and
means for, at least once, after attracting the magnetizable or magnetic objects to the sensor surface, measuring the amount of magnetizable or magnetic objects attached to the sensor surface by using the sensor element.

14. A system according to claim 13, furthermore comprising:
means for monitoring the amount of attached magnetizable or magnetic objects to the sensor surface as a function of time.
